(19) United States
(12) Patent Application Publication
(10) Pub. No.: US 2025/0331924 A1
(43) Pub. Date: Oct. 30, 2025

(54) BI-PLANE/MULTI-VIEW FLUOROSCOPIC FUSION VIA EXTENDED REALITY SYSTEM

(71) Applicant: MEDIVIEW XR, INC., Cleveland, OH (US)

(72) Inventors: Jeffrey Harold Yanof, Solon, OH (US); Adam E. Rakestraw, Findlay, OH (US); Gaurav Gadodia, Chicago, IL (US); Aydan Thomas Hanlon, Akron, OH (US); Peter Nicholas Braido, Wyoming, MN (US); Ross Daniel Hinrichsen, Plymouth, MN (US)

(21) Appl. No.: 19/191,221

(22) Filed: Apr. 28, 2025

Related U.S. Application Data
(60) Provisional application No. 63/639,115, filed on Apr. 26, 2024.

(51) Int. Cl. A61B 34/10; A61B 34/20; A61B 90/00; G02B 27/01; G06T 19/00

(52) U.S. Cl. CPC A61B 34/10; A61B 34/20; A61B 90/361; G02B 27/0172; G06T 19/006; A61B 2034/105; A61B 2034/2055; A61B 2090/365; G02B 2027/0174

(57) ABSTRACT

A system and method for performing a medical procedure on a location of interest of a patient includes a first imaging system, a second imaging system, a computer system, and an augmented reality display system. The first imaging system can be configured to acquire a first image and the second imaging system can be configured to acquire a second image of the location of interest. The computer system can be configured to register the first image and the second image, establish a spatial correlation between the first image, the second image, and the patient, and generate a holographic visualization combining the first image and the second image. The augmented reality display system can be configured to project the holographic visualization and align the first image and the second image with the patient.

### CROSS-REFERENCE TO RELATED APPLICATIONS

This application claims the benefit of U.S. Provisional Application No. 63/639,115, filed on Apr. 26, 2024. The entire disclosure of the above application is incorporated herein by reference.

### FIELD

The present technology relates to an augmented reality system and, more specifically, to an augmented reality system for use during a surgical procedure.

### INTRODUCTION

This section provides background information related to the present disclosure which is not necessarily prior art.

The field of medical imaging has aided healthcare by enabling a physician to non-invasively visualize internal body structures. From basic X-rays to advanced imaging techniques like computed tomography (CT), magnetic resonance imaging (MRI), and fluoroscopy, imaging technology can be a helpful tool for diagnosing conditions and guiding surgical intervention. The evolution of medical imaging has transformed the way the physician can approach patient care, moving from largely invasive diagnostic procedures to non-invasive visualization methods. Medical imaging has allowed the medical professional to detect and diagnose certain conditions with increasing precision and accuracy.

Fluoroscopy, which functions as a real-time X-ray video stream, has emerged as a technology in operating rooms and interventional radiology suites. The imaging capability of fluoroscopy can assist the practitioner during a wide spectrum of minimally invasive procedures, including the ability to provide precise navigation through an anatomical structure. The ability to visualize internal structures in real-time has enabled the development of numerous minimally invasive surgical techniques. In turn, the growth of minimally invasive procedures has been driven by benefits including reduced patient recovery time and improved surgical outcomes.

Despite advances in medical imaging technology, challenges persist in surgical procedures that rely on fluoroscopic guidance. One limitation relates to the practitioner having to mentally reconstruct three-dimensional anatomical relationships while working with two-dimensional fluoroscopic images. The cognitive burden can become pronounced during a complex intervention, potentially leading to an extended procedure time and increased radiation exposure for both the patient and medical staff. The complexity of the mental mapping process can also impact the precision and efficiency of a surgical procedure.

The ergonomic constraints of a fluoroscopic technique presents additional challenges in the operating environment. The practitioner must frequently shift attention between the patient and a remotely positioned fluoroscopic monitor, disrupting procedural workflow and creating physical strain over an extended period of time. Further, a fixed monitor position can result in a suboptimal viewing angle that can complicate surgical navigation. The physical arrangement of equipment in the operating room can create awkward positioning requirements for the surgical team. The constant need to adjust position and attention between the patient and the imaging display impacts both procedure efficiency and practitioner comfort.

Accordingly, there is a need for an augmented reality system that can provide a real time bi-plane/multi-view fluoroscopic fusion hologram rendered in proximity to a patient for use during a procedure.

### SUMMARY

In concordance with the instant disclosure, a need for an augmented reality system that can provide a real time bi-plane/multi-view fluoroscopic fusion hologram rendered in proximity to a patient for use during a procedure, has surprisingly been discovered.

The present technology includes articles of manufacture, systems, and processes that relate to the use of augmented reality and at least one imaging system during a medical procedure, including systems and methods for registering and projecting real-time bi-plane and multi-view fluoroscopic fusion holograms rendered in proximity to a patient, enabling a practitioner to simultaneously visualize a fluoroscopic image, an interventional instrument, and patient anatomy while maintaining precise spatial registration and reducing radiation exposure.

In certain embodiments, a system for performing a medical procedure on a location of interest of a patient can include a first imaging system, a second imaging system, a computer system, and an augmented reality display system. The first imaging system can be configured to acquire a first image of the location of interest. The second imaging system can be disposed non-coplanar to the first imaging system and can be configured to acquire a second image of the location of interest. The computer system can be configured to register the first image and the second image using the anatomical registration device, establish a spatial correlation between the first image, the second image and patient, and generate a holographic visualization combining the first image and the second image. The augmented reality display system can be configured to project the holographic visualization in proximity to the patient, spatially align the first image and the second image with the patient, and enable simultaneous visualization of the first image, the second image, and the patient.

In certain embodiments, a method for performing a medical procedure at a location of interest on a patient is provided. The method can include providing the system for performing a medical procedure on a location of interest of a patient as described herein. The first imaging system can acquire the first image, and the second imaging system can acquire the second image. The second image can depict a different view of the location of interest than the first image. The first image and the second image can be registered to establish a spatial correlation between the first image, the second image, and the patient. The method can include generating a holographic visualization that combines the first image, the second image, and a virtual trajectory for an interventional instrument. The holographic visualization can be projected in proximity to the patient such that the first image and the second image are spatially aligned with the patient. The orientation and depth of the interventional instruction can be adjusted using the holographic visualization. The method can include performing the medical procedure guided by the spatially aligned holographic visualization.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic of a system for performing a medical procedure on a location of interest of a patient;

FIG. 2A is a schematic depicting a use case in which an interventional device is not initially aligned down-the-barrel of a C-arm fluoroscopy central ray;

FIG. 2B is a schematic depicting a use case in which an interventional device is adjusted by a practitioner to achieve alignment;

FIG. 3 is a schematic illustrating two fluoroscopy image streams disposed perpendicular to each other;

FIG. 4 is an environmental view of a holographic visualization generated by the system for performing a medical procedure on a location of interest of a patient during a procedure;

FIG. 5 is a schematic depicting a process to register a multidetector row CT (MDCT) data set in the coordinate system of a bi-planar C-arm; and

FIGS. 6A-6C provide a flowchart depicting a method for performing a medical procedure at a location of interest on a patient.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the term “head-mounted device” or “headset” or “HMD” refers to a display device, configured to be worn on the head, that has one or more display optics (including lenses) in front of one or more eyes. These terms may be referred to even more generally by the term “augmented reality system,” although it should be appreciated that the term “augmented reality system” is not limited to display devices configured to be worn on the head. In some instances, the head-mounted device can also include a non-transitory memory and a processing unit. An example of a suitable head-mounted device is a Microsoft HoloLens®.

Additionally, non-head mounted devices can be used similarly such as a pass-through phone, tablet, or screen, as examples. It should be appreciated that projected images, like AR Projectors, can be shown in different modalities.

As used herein, the terms “imaging system,” “image acquisition apparatus,” “image acquisition system” or the like refer to technology that creates a visual representation of the interior of a body of a patient. For example, the imaging system can be a computed tomography (CT) system, a fluoroscopy system, a magnetic resonance imaging (MRI) system, an ultrasound (US) system, or the like.

As used herein, the terms “coordinate system” or “augmented realty system coordinate system” refer to a 3D Cartesian coordinate system that uses one or more numbers to determine the position of points or other geometric elements unique to the particular augmented reality system or image acquisition system to which it pertains. For example, the headset coordinate system can be rotated, scaled, or the like, from a standard 3D Cartesian coordinate system.

As used herein, the terms “image” or “image data” or “image dataset” or “imaging data” refers to information recorded in 3D by the imaging system related to an observation of the interior of the patient's body. For example, the “image data” or “image dataset” can include processed two-dimensional or three-dimensional images or models such as tomographic images, e.g., represented by data formatted according to the Digital Imaging and Communications in Medicine (DICOM) standard or other relevant imaging standards.

As used herein, the terms “imaging coordinate system” or “image acquisition system coordinate system” refers to a 3D Cartesian coordinate system that uses one or more numbers to determine the position of points or other geometric elements unique to the particular imaging system. For example, the imaging coordinate system can be rotated, scaled, or the like, from a standard 3D Cartesian coordinate system.

As used herein, the terms “hologram,” “holographic,” “holographic projection,” “holographic representation,” or “holographic visualization” refer to a computer-generated image projected to a lens of a headset. Generally, a hologram can be generated synthetically (in an augmented reality (AR)) and is not related to physical reality.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two things being linked or coordinated. For example, a coil-sensor can be integrated with an interventional device.

As used herein, the term “degrees-of-freedom” or “DOF” refers to a number of independently variable factors. For example, a tracking system can have six degrees-of-freedom (or 6DOF), a 3D point and 3 dimensions of rotation.

As used herein, the term “real-time” refers to the actual time during which a process or event occurs. In other words, a real-time event is done live (within milliseconds so that results are available immediately as feedback). For example, a real-time event can be represented within 100 milliseconds of the event occurring.

As used herein, the terms “subject” and “patient” can be used interchangeably and refer to any vertebrate organism.

As used herein, the term “registration” refers to steps of transforming tracking data and body image data to a common coordinate system and creating a holographic display of images and information relative to a body of a physical patient during a procedure.

As used herein, the terms “interventional device”, “tracked instrument”, or “interventional instrument” refers to a medical instrument used during the medical procedure. For example, the interventional instrument can include a needle, an ablation probe, a catheter, a stent, and a surgical tool.

As used herein, the term “C-arm system” or “C-arm apparatus” refers to a C-Arm—Fluoroscopy Machines having a C-arm and an imaging frustrum. An example C-arm system is the OEC Elite CFD, which is commercially available from General Electric (Boston, MA).

As used herein, the term “location of interest” on a patient refers to an anatomical site where a medical procedure is performed. The location of interest can encompass areas requiring orthopedic procedures such as knee, hip, shoulder, hand/wrist, foot/ankle and spine interventions. For neurological procedures, the location can include areas involving high- and low-grade gliomas, metastases, astrocytomas, abscess drainage sites, hematoma locations, pituitary adenomas, clival chordomas, meningiomas, and craniopharyngiomas. In pain management applications, the location of interest can include sites for nerve blocks and ablations, such as femoral and obturator nerves, genicular nerves, medial branch nerves, vertebral areas for vertebroplasty/vertebral augmentations, and regions requiring epidural injections. For cardiovascular procedures, the location of interest can include areas involving transcatheter valve and stent placement. It should be appreciated that the location of interest can include an anatomical registration device to establish spatial correlation between imaging views and the physical anatomy of the patient.

As used herein, the term “practitioner” refers to any medical professional including, but not limited to, surgeons, physicians, doctors, nurses, and support staff who are physically or remotely present.

As used herein, the term spatial “registration” refers to steps of transforming tracking and imaging dataset associated with virtual representation of tracked devices—including holographic guides, applicators, and ultrasound image stream—and additional body image data for mutual alignment and correspondence of said virtual devices and image data in the head mounted displays coordinate system enabling a stereoscopic holographic projection display of images and information relative to a body of a physical patient during a procedure, for example, as further described in U.S. Pat. No. 10,895,906 to West et al., and also applicant's co-owned U.S. patent application Ser. No. 17/110,991 to Black et al. and U.S. Pat. No. 11,701,183 to Martin III et al., the entire disclosures of which are incorporated herein by reference.

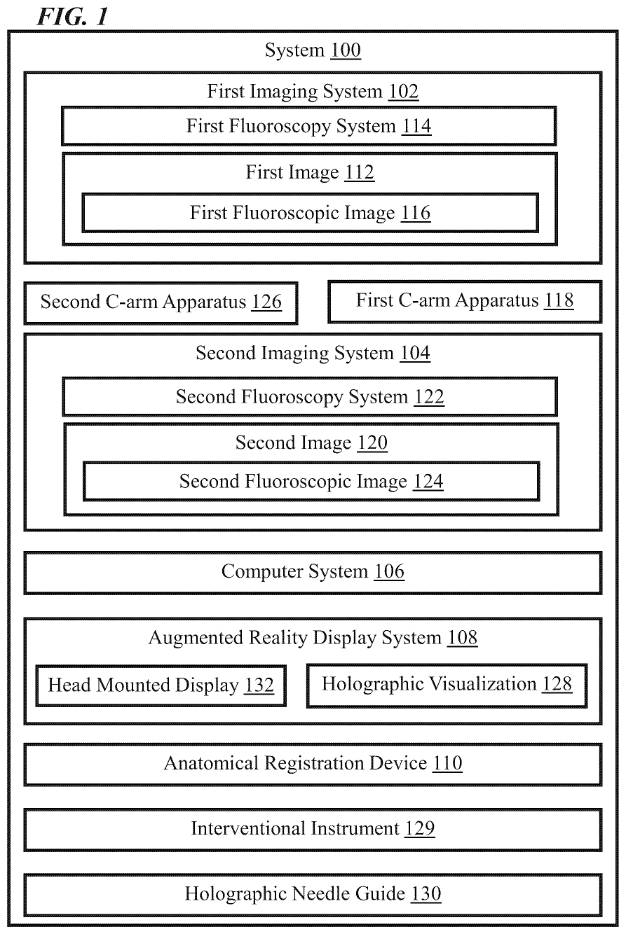

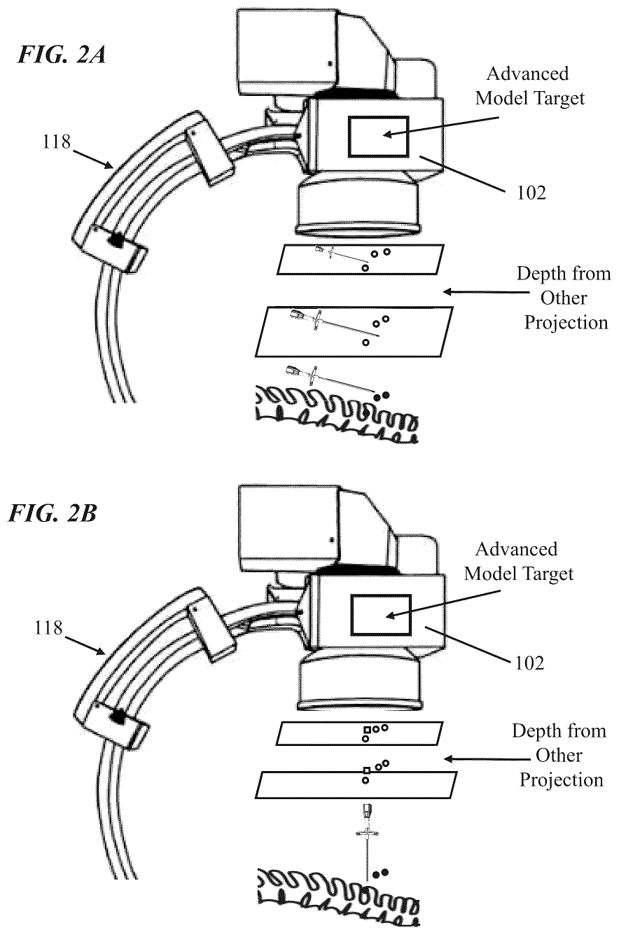

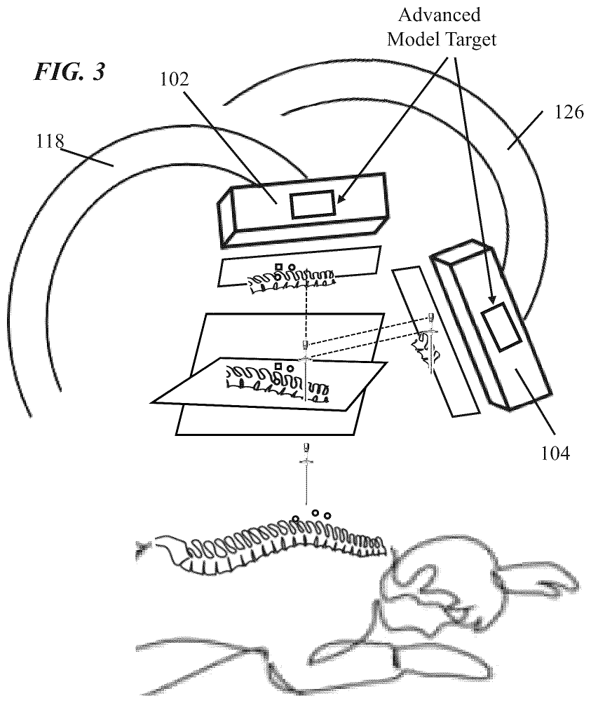

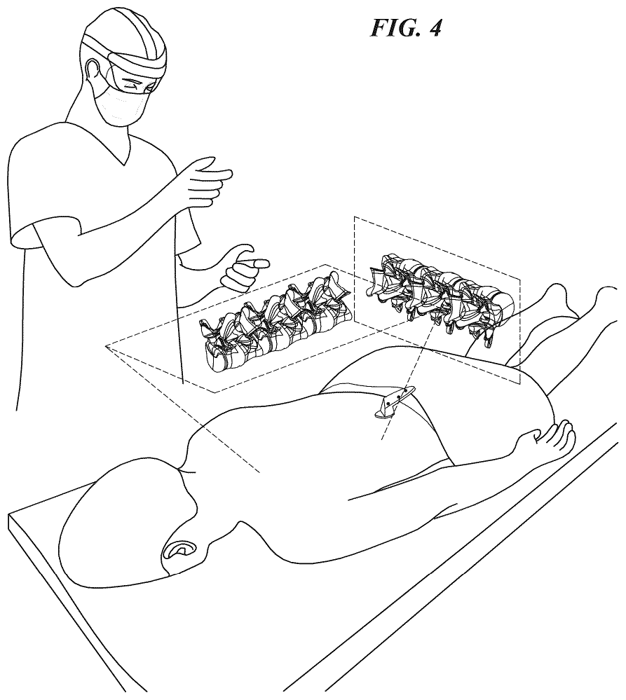

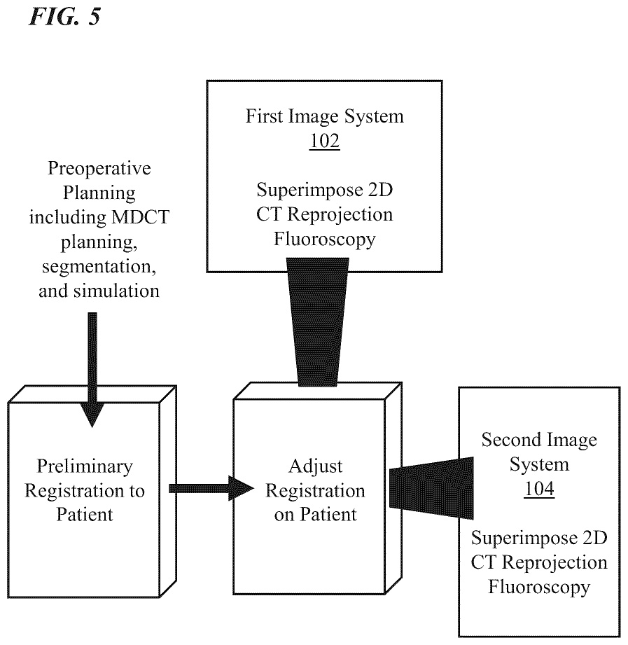

The present technology relates to ways of performing a procedure on a patient utilizing an augmented reality system, shown generally in FIGS. 1-2. An embodiment of a system 100 for performing a procedure on a patient by a practitioner is shown in FIG. 1. The system 100 can be configured to use one or more perspective projections to augment a set of virtual objects derived from imaging systems to allow the practitioner to project and cross-reference images from multiple imaging systems. The system 100 for performing a procedure on a patient by a practitioner can include a first imaging system 102, a second imaging system 104, a computer system 106, and an augmented reality display system 108.

The first imaging system 102 can be configured to acquire a first image 112 of the location of interest. The first imaging system 102 can include a first fluoroscopy system 114 configured to acquire a first fluoroscopic image 116. In an alternative embodiment, the first imaging system 102 can include a multidetector row CT (MDCT), a cone beam CT (CBCT), and a PET scanner. The first imaging system 102 can be configured to work with both 2D and 3D imaging modalities, allowing for the acquisition of 2D fluoroscopic projections as well as 3D volumetric datasets. For a procedure requiring real-time imaging, the system 100 can include a mono-planar or a bi-planar fluoroscopy configuration. The system 100 can also be integrated with ultrasound imaging systems and electromagnetic navigation systems for comprehensive procedural guidance. Additionally, the first imaging system 102 can be configured to work with a pre-operative imaging dataset and can incorporate capabilities for image fusion and registration with other imaging modalities. A skilled artisan can select a suitable first imaging system 102 within the scope of the present disclosure.

The first imaging system 102 can work in conjunction with the second imaging system 104. The second imaging system 104 can be configured to acquire a second image 120 of the location of interest. The second imaging system 104 can include a second fluoroscopy system 122 configured to acquire a second fluoroscopic image 124. In an alternative embodiment, the second imaging system 104 can include a multidetector row CT (MDCT), a cone beam CT (CBCT), and a PET scanner. The second imaging system 104 can be configured to work with both 2D and 3D imaging modalities, allowing for the acquisition of 2D fluoroscopic projections as well as 3D volumetric datasets. For a procedure requiring real-time imaging, the system 100 can include a mono-planar or a bi-planar fluoroscopy configuration. The system 100 can also be integrated with ultrasound imaging systems and electromagnetic navigation systems for comprehensive procedural guidance. Additionally, the second imaging system 104 can be configured to work with a pre-operative imaging dataset and can incorporate capabilities for image fusion and registration with other imaging modalities. A skilled artisan can select a suitable second imaging system 104 within the scope of the present disclosure.

It should be appreciated that the first image 112 collected by the first imaging system 102 and the second image 120 acquired by the second imaging system 104 can include a 2D image or a 3D image. In an embodiment where the first imaging system 102 includes the first fluoroscopy system 114 or the second imaging system 104 includes a second fluoroscopy system 122, the first image 112 or the second image 120 can include a 2D fluoroscopic image. For example, the first imaging system 102 and the second imaging system 104 can be positioned relative the patient such that the first image 112 can include an anteroposterior view or a lateral view of the patient. Where a multidetector row CT (MDCT), a cone beam CT (CBCT), or a PET scanner is utilized as a first imaging system 102 or the second imaging system 104, the first image 112 or the second image 120 can include 3D volumetric data that can be used for perspective reprojection and image fusion. The first image 112 and the second image 120 can also include pre-operative imaging data that can be registered and aligned with a real-time 2D fluoroscopic image during the procedure. It should be appreciated that in the procedure using a bi-planar fluoroscopy, the first image 112 and the second image 120 can include more than one 2D view that can be combined to provide a spatial orientation and depth perception when displayed through the augmented reality display system 108.

As described herein, the first imaging system 102 can be positioned to acquire an anteroposterior (AP) fluoroscopic image of the location of interest on the patient. The position of the first imaging system 102 can be adjusted and aligned with an anatomical registration device 110 to optimize the collection of the first image 112, in certain embodiments. The first imaging system 102 can be mounted on a first C-arm apparatus 118, which can allow for rotational movement and repositioning to capture different viewing angles and orientations. The first imaging system 102 can be integrated into the first C-arm apparatus 118. The C-arm configuration can enable the practitioner to adjust the position of the first imaging system 102 between the anteroposterior view and the lateral view.

As described herein, the second imaging system 104 can be positioned to acquire a lateral fluoroscopic image of the location of interest on the patient. The position of the second imaging system 104 can be adjusted and aligned with the anatomical registration device 110 to optimize the collection of the second image 120. The second imaging system 104 can be mounted on a second C-arm apparatus 126, which can allow for rotational movement and repositioning to capture different viewing angles and orientations. The second imaging system 104 can be integrated into the second C-arm apparatus 126. The C-arm configuration can enable the practitioner to adjust the position of the second imaging system 104 between the anteroposterior view and the lateral view.

The first fluoroscopy system 114 and the second fluoroscopy system 122 can be positioned non-coplanar to each other via the first C-arm apparatus 118 and the second C-arm apparatus 126 to enable acquisition of multiple views from different angles. In a particular example, the second fluoroscopy system 122 can be disposed perpendicular to the first fluoroscopy system 114. The bi-planar configuration can allow for simultaneous acquisition of anteroposterior and lateral fluoroscopic images of the location of interest. When configured in the perpendicular arrangement, the systems can provide spatial orientation and depth perception by combining the two different viewing angles. The positioning of the first fluoroscopy system 114 and the second fluoroscopy system 122 can be tracked using the anatomical registration device 110 in a coordinate system compatible with the augmented reality display system 108 to maintain proper spatial registration. It should be appreciated that the first C-arm apparatus 118 and the second C-arm apparatus 126 can be rotated sequentially to capture the different perspectives, typically within a few seconds of each other. A skilled artisan can select a suitable position for the first C-arm apparatus 118 and the second C-arm apparatus 126 within the scope of the present disclosure.

The computer system 106 can include a processor and a memory. The computer system 106 can be in communication with the augmented reality display system 108, the first imaging system 102, and the second imaging system 104. The computer system 106 can be configured by machine-readable instructions to register the first image 112 and the second image 120 and establish a spatial correlation between the first image 112, the second image 120, and patient. The spatial correlation can be established through preliminary registration of a CT image to the using the anatomical registration device 110. The initial registration can be refined through respiratory phase matching and breath/ventilation techniques to account for the rhythmic movement of the patient when breathing. Rhythmic movement of the patient can be tracked, for example, as described in co-owned U.S. patent application Ser. No. 17/203,728 to Black et al., the entire disclosure of which is incorporated herein by reference.

It should be appreciated that for fluoroscopic imaging via the first fluoroscopy system 114 and the second fluoroscopy system 122, the computer system 106 can perform perspective reprojection of the image volume along the optical access of the first C-arm apparatus 118 and the second C-arm apparatus 126 to create 2D virtual displays that can be fused or superimposed with a live fluoroscopic image. The system 100 can allow for adjustment of 3D rotation and translation to align fluoroscopic images with reprojected images.

The spatial correlation process can also include automatic segmentation of skin and bone surfaces from the first image 112 and the second image 120, which can aid in establishing an anatomical landmark for registration. The system 100 can incorporate a radio-opaque CT fiducial marker and support transformation of the CT fiducial marker to coordinates of the augmented reality display system 108 for maintaining spatial alignment.

The computer system 106 can generate a holographic visualization 128 combining the first image 112 and the second image 120. The holographic visualization 128 generated by the computer system 106 can combine the first image 112, second image 120, and a virtual trajectory for an interventional instrument 129. The augmented reality display system 108 can project the holographic visualization 128 in proximity to the patient such that the first image 112 and second image 120 are spatially aligned with the patient. The holographic visualization 128 can enable the practitioner to simultaneously view fluoroscopic images, imaged interventional instruments, and the patient without turning toward a physical 2D monitor. When using bi-planar fluoroscopy, the holographic visualization 128 can project two perpendicular views that maintain correlation with the body of the patient, with the fluoroscopic image streams formed by the perspective projection of the C-arm imaging chain.

For registration, the computer system 106 can process and perform automatic segmentation of skin and bone surfaces from the first image 112 and the second image 120 and can incorporate surgical planning data into the anatomical visualizations. The computer system 106 can support real-time tracking, including the ability to track a position of the interventional instrument 129 relative to the first image 112, the second image 120, and the patient, and update the holographic visualization 128 to show the tracked position in real-time. The computer system 106 can also perform respiratory phase matching and analysis to improve registration accuracy during procedure.

Turning now to the augmented reality display system 108, which can stereoscopically project the holographic visualization 128 through see-through lenses using video pass-through, as shown in FIG. 2. The augmented reality display system 108 can include a head-mounted display 132 configured to project the holographic visualization 128. When displaying the first image 112 and/or the second image 120, the augmented reality display system 108 can project the holographic visualization 128. The augmented reality display system 108 can be similar to the augmented reality system disclosed in U.S. Pat. No. 11,967,036 to Black and U.S. patent application Ser. No. 17/505,772 to Black, each incorporated herein by reference.

The holographic visualization 128 can be viewed from a broad range of viewing angles and ergonomic positions while navigating the interventional instrument 129. The augmented reality display system 108 can also project a supplemental view and rotate the holographic visualization 128 relative to the viewing direction of the practitioner via a controlled angle. In this way, hand-eye coordination of the practitioner can be improved and maintained.

The holographic visualization 128 can include an adjustable holographic needle guide 130 projected as an adjustable line segment representing an instrument guide path. The holographic needle guide 130 can be projected as an adjustable line segment representing an instrument guide path for the interventional procedure. The adjustable holographic needle guide 130 can be used in conjunction with the bi-planar holographic projection to plan and execute one or more instrument trajectories. Specifically, the holographic needle guide 130 can be aligned with a central line of the first C-arm apparatus 118 and perpendicular to the second C-arm apparatus 126 to plan depth during the procedure.

The holographic needle guide 130 can be dynamically adjusted and updated in real-time as the computer system 106 tracks the position of the interventional instrument 129. Advantageously, the practitioner can visualize and adjust the orientation and depth of the interventional instrument 129 using the holographic visualization 128 during procedures based on the adjustable holographic needle guide 130. The adjustable holographic needle guide 130 can work as part of the broader holographic visualization system that enables the practitioner to maintain hand-eye coordination while simultaneously viewing fluoroscopic images, imaged interventional instruments, and the patient.

The anatomical registration device 110 can be configured to be positioned on the location of interest of the patient. The anatomical registration device 110 can enable registration of the first image 112 and the second image 120 to establish the spatial correlation between the first image 112, the second image 120, and the patient. The anatomical registration device 110 can include a sensor and tracking component. For example, the anatomical registration device 110 can include a sensor such as a fiducial sensor, an electromagnetic sensor, an inertial measurement sensor, an optical sensor, an infrared sensor, an image target, an acoustic sensor, and combinations thereof. A skilled artisan can select a suitable anatomical registration device 110 within the scope of the present disclosure.

The anatomical registration device 110 can include an alignment indicator for aligning with a midline of the patient. The anatomical registration device 110 can include multiple tracking modalities, including a 2D Image Targets (IT), an 3D Advanced Model Target (AMT), and an active LED infrared (IR) sensor, which can include one or more inertial measurement unit (IMU) sensors to bridge a line-of-sight obscuration.

It should be appreciated that the AMT can include a registration device for enhanced accuracy and flexibility in a medical procedure. Unlike a 2D image target, an AMT can include multiple distinct features around the entire exterior surface of the AMT that enables recognition from any viewing angle. The AMT can be effectively utilized to track the position and orientation of the first C-arm apparatus 118 and the second C-arm apparatus 126 during the fluoroscopic procedure. The system 100 can track the C-arm pose (mono- or bi-plane) using an image or model target in head mounted display (HMD) world coordinates.

For bi-planar configurations, the AMT can help track both the first C-arm apparatus 118 and the second C-arm apparatus 126, as the first fluoroscopic image 116 and the second fluoroscopy system 122 can be positioned non-coplanar to each other via the respective C-arm apparatuses 118, 126 to enable acquisition of multiple views from different angles. The positioning of both the first fluoroscopic image 116 and the second fluoroscopy system 122 can be tracked using the registration device in a coordinate system compatible with the augmented reality system to maintain proper spatial registration. As described herein, the system 100 can utilize several types of registration techniques such as 2D Image Targets (IT), 3D Advanced Model Targets (AMT), and active LED infrared (IR) sensors. The system 100 can also include Inertial Measurement Units (IMUs) to overcome line-of-sight limitations. The system 100 can utilize a registration device to establish a registration of the virtual representation of the physical interventional instrument 129 using the imaged anatomy of a location of interest on the patient. This can be achieved by creating a digital twin or a time series data stream that correlates with the first image 112, the second image 120 and any related image data in near-real time. The system 100 can be focused on applications that require precise navigation and guidance, such as orthopedic, neuro, pain management, and cardiovascular procedures.

In practical applications, the system 100 operates by first capturing pre-procedure or intra-procedure data, such as from MDCT, CBCT, or PET scans, which can include radio-opaque CT fiducial markers for optional respiratory phase matching. The spatial registration workflow of the system 100 can involve capturing image targets on cone beam CT with the augmented reality display system 108, which transforms the model target, ARD, or optical image targets to headset coordinates of the augmented reality display system 108. The spatial registration workflow can allow for the tomographic-based anatomy, live imaging (ultrasound or fluoroscopy) including the first image 112 and the second image 120, and a tracked light ray hologram or adjustable holographic needle guide 130 to be registered as a perspective projection to the patient, enhancing the accuracy and efficacy of the medical procedure.

As described hereinabove, the second imaging system 104 can include mono or biplanar holographic projection system. The system 100 can steam a live 2D fluoroscopy image stream near the patient aligned with the physical orientation of the patient. In this way, the left, right, anterior, and posterior sides of the image correspond directly with the respective sides of the patient. The spatial correlation between the live images 112, 120 and the anatomy of the patient aids with maintaining intuitive eye-hand coordination during a procedure. Advantageously, the system 100 can allow the practitioner to view the fluoroscopic images 112, 120 and the patient simultaneously without the need to divert their gaze to a separate 2D monitor, thereby streamlining the workflow and potentially reducing procedure times.

The biplanar holographic projection aspect of the system allows 100 for the stereoscopic projection of two fluoroscopic image streams 112, 120 that are acquired and displayed in orientations that are approximately perpendicular to each other, which can allow for the practitioner to maintain a correlation with the physical body, allowing the practitioner to adjust the orientation and depth of interventional device with improved precision.

It should be appreciated that the system 100 can enhance the guidance and navigation for the placement of the interventional instrument 129 by utilizing fiducial markers placed on the patient. The fiducial markers can work in conjunction with the projection of mutually perpendicular fluoroscopic views 112, 120 rendered by the augmented reality display system 108, which can be oriented in proximity to the patient. The ability of the augmented reality display system 108 to update the stereoscopic holographic visualization 128 according to the live fluoroscopic views 112, 120 allows for precise planning and adjustment of the adjustable holographic needle guide 130 or other devices with further features include being luminal, flexible, or laparoscopic, as examples. The adjustable holographic needle guide 130 can be projected as an adjustable line segment representing the guide path of the interventional instrument 129. Advantageously, the system 100 can enhance the way practitioners interact with imaging data 112, 120, providing a more intuitive and efficient method for conducting a minimally invasive procedure.

With respect to the registration process, using a Multidetector Row Computed Tomography (MDCT) in a biplanar system can facilitate the accuracy of the medical imaging by integrating MDCT data with live fluoroscopic imaging 112, 120. The system 100 can automatically segment the MDCT dataset into distinct anatomical structures such as skin and bone surfaces. A surgical plan can be simulated using preoperative data such as the MDCT dataset, and the results, including the positions and trajectories of instruments or implants, can be projected alongside the anatomical data set for pre-procedural planning.

To achieve registration, the holographic projections of the anatomical structures derived from the MDCT can be initially aligned with the physical patient using the HMD. This coarse registration can be refined by adjusting the holographic projections using translation and rotation controls on a bounding box that encompasses the image volume. The scale of the original image data is maintained during this process. The registration can be further refined by reprojecting live fluoroscopic images according to the geometry of the C-arm imaging chain. This allows for the display of digitally reconstructed fluoroscopic views to be compared with the live fluoroscopic images. In the case of a biplane system, two sets of projections—one for lateral and one for anteroposterior (AP) views—can be used. These projection pairs can include live fluoroscopic views and static reprojections of the MDCT data. By comparing these pairs, the practitioner can make fine adjustments to the rotation and translation of the MDCT dataset, ensuring that the MDCT data is accurately registered with the anatomy of the patient in the biplanar system. Desirably, the precise alignment can contribute to successful execution of image-guided medical procedures.

It should also be appreciated the system 100 can utilize any number of imaging system and/or images for the holographic visualization 128. The system 100 can register any number of images using the anatomical registration device 110 to establish a spatial correlation between the images and the patient. The system 100 can generate the holographic visualization 128 based on any number of images and project the holographic visualization 128. A skilled artisan can select a suitable number of imaging systems and images within the scope of the present disclosure.

Advantageously, the system 100 can enable the practitioner to simultaneously view the first image 112 and the second image 120, the interventional instrument 129, and the patient without turning toward a physical 2D monitor, improving positioning and ergonomics. The spatial orientation and perception can allow for faster procedures by minimizing time to target and militating against the need for repositioning. The system 100 can support the minimally invasive procedure while maintaining precision and accuracy, helping to address rising healthcare costs and medical staff burnout through improved efficiency.

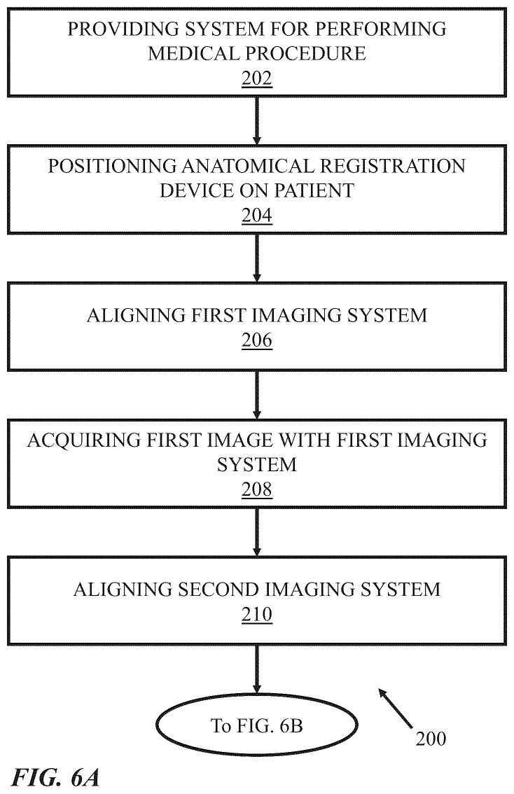

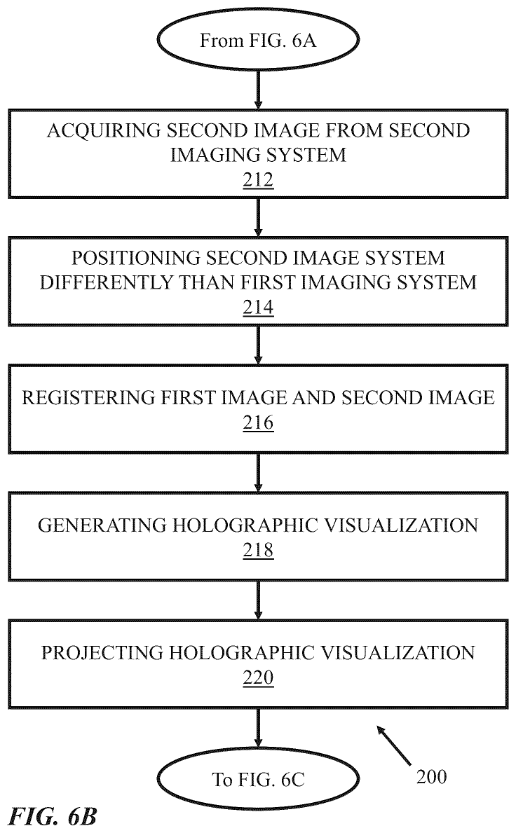

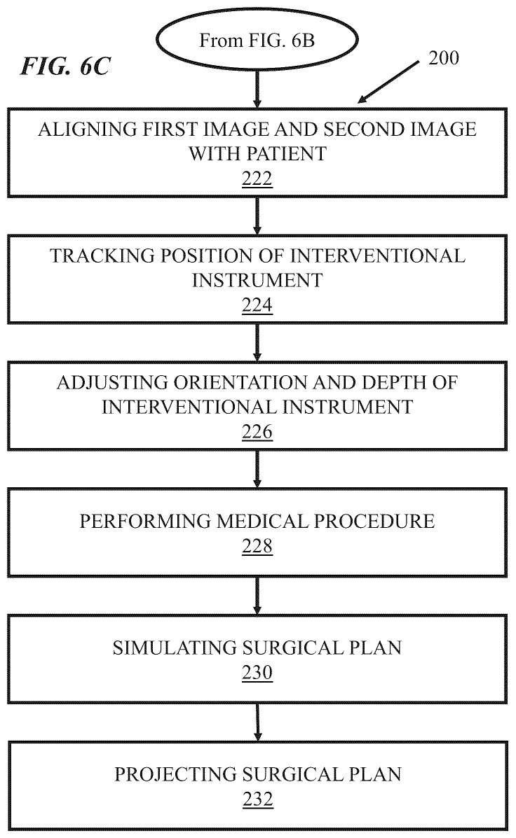

The present disclosure provides a method 200 for performing a medical procedure at a location of interest on a patient, shown in FIGS. 6A-6C. For example, the medical procedure can include an orthopedic procedure, a neurological procedure, a pain management procedure, an otolaryngology procedure, or a cardiovascular procedure. A skilled artisan can select a suitable medical procedure within the scope of the present disclosure. The method 200 can include a step 202 of providing the system 100 as described herein.

In a step 204, the anatomical registration device 110 can be positioned on the location of interest of the patient. The method 200 can include a step 206 of aligning the first imaging system 102 to collect the first image 112. For example, the first imaging system 102 can be aligned to facilitate the first image 112 to include an anteroposterior fluoroscopic image of the location of interest. The first imaging system 102 can acquire the first image 112 depicting a first view of the location of interest in a step 208.

The method 200 can include a step 210 of aligning the second imaging system 104 to collect the second image 120. For example, the second imaging system 104 can be aligned to facilitate the second image 120 to include a lateral fluoroscopic image of the location of interest. The second imaging system 104 can acquire the second image 120 a second view of the location of interest in a step 212. As described herein, the second imaging system 104 can be positioned at a different angle relative the patient than the first imaging system 102 such that the second view can be different than the first view in a step 214.

In a step 216, the first image 112 and the second image 120 can be registered using the anatomical registration device 110 to establish a spatial correlation between the first image 112, the second image 120, and the patient. The holographic visualization 128 can be generated that combines first image 112, the second image 120, and a virtual trajectory for the interventional instrument 129 in a step 218. In a step 220, the holographic visualization 128 can be projected in proximity to the patient such that the first image 112 and the second image are 120 spatially aligned with the patient and viewed through the augmented reality display system 108. The step 220 of projecting the holographic visualization includes a step 222 of aligning the first image 112 and the second image 120 such that a first anterior side and a first posterior side of the first image and a second anterior side and a second posterior side of the second image correspond with a patient anterior side and a patient posterior side of the patient.

The method 200 can include a step 224 of tracking a position of the interventional instrument 129 using the anatomical registration device 110 during the medical procedure. The orientation and depth of the interventional instrument can be adjusted using the holographic visualization 128 in a step 226. The method 200 can include a step 228 of performing the medical procedure guided by the spatially aligned holographic visualization.

It should be appreciated that the method 200 can include a step 230 of simulating a surgical plan and a step 232 of projecting the surgical plan onto the patient using the augmented reality display system. The surgical plan can be simulated with the MDCT dataset and can include planning the position and trajectory of interventional instrument 129 or an implant, which can be projected with the anatomical data set as a holographic visualization. For procedures like arthrodesis surgeries, the surgical plan can include graft placement planning and screw/plate placement planning that can be used for intraprocedural guidance. The system 100 can allow for pre-operative planning where the practitioner can select the patient dataset and review reports on related cohort data. The surgical plan can be refined through registration and alignment processes, where the MDCT-based dataset is automatically segmented into skin and bone surfaces and registered to the physical patient using the anatomical registration device. For specific procedures like arthroplasty, the surgical place can include detailed steps for implant placement planning, such as positioning of balls, sockets, and stems, as well as pre- and post-operative assessments of anatomical structures and their relationships to planned hardware placement. The surgical plan can be adjusted during the procedure using real-time fluoroscopic imaging 112, 120 that is spatially aligned with the pre-operative plan through the augmented reality system.

### EXAMPLES

The following examples demonstrate embodiments of the present disclosure in use. The examples are provided for illustrative purposes only and should not be construed as limiting the scope of the present disclosure. It will be appreciated by those skilled in the art that various modifications, alternatives, and variations of the examples can be made without departing from the spirit and scope of the present disclosure as defined by the appended claims.

In a first exemplary application, the system can be utilized for an orthopedic procedure, specifically for pedicle screw insertion during spinal surgery. The system 100 can be configured to assist with spinal level localization and guide pedicle screw insertion. For the procedure, the first imaging system 102 can be positioned to acquire anteroposterior fluoroscopic images while the second imaging system 104 can be positioned perpendicular to capture lateral views of the spine. The anatomical registration device 110 can be placed on the back of the patient aligned with the spinous process using the midline indicator to establish proper orientation.

The computer system 106 registers both fluoroscopic views using the anatomical registration device to establish a spatial correlation. The augmented reality display system 108 can project the holographic visualization 128 combining the anteroposterior and lateral fluoroscopic views, spatially aligned with the patient. The bi-planar visualization can enable the practitioner to simultaneously view both perspectives while maintaining hand-eye coordination, militating against the need to repeatedly look away at 2D monitors.

For pedicle screw placement, the system 100 can project an adjustable holographic needle guide 130 representing the planned trajectory. The adjustable holographic needle guide 130 can be aligned with the central ray of the first C-arm apparatus 118 while being perpendicular to the second C-arm apparatus 126 to plan screw depth and trajectory. The system 100 can track the position of the interventional instrument 129 in real-time, updating the holographic visualization 128 to show the relationship between the interventional instrument 129 and the anatomical structures of the patient.

The system 100 can provide advantages for the orthopedic procedure by reducing radiation exposure by imaging acquisition, improving accuracy through spatial orientation and real-time guidance, and decreasing procedure time by eliminating the need to repeatedly reposition the C-arm between anteroposterior and lateral views. The collaborative features also enable remote procedural mentoring, allowing the practitioner to provide guidance through shared holographic visualizations. Additionally, the system 100 can captures procedural data for post-operative analysis, enabling comparison of planned versus actual screw placement and trajectories to help optimize future procedures.

In a second exemplary application, the system 100 can be configured to assist with identifying a brain shift after pre-operative scanning and determining an extent of tumor resection. For the procedure, the first imaging system 102 and second imaging system 104 can be positioned non-coplanar to each other to acquire multiple views of the surgical site. The anatomical registration device 110 can be positioned on the location of interest to establish spatial correlation between the pre-operative images and the anatomy of the patient, helping account for any brain shift that has occurred.

During tumor resection, the system 100 can enable the practitioner to monitor resection progress and assess the residual tumor in real-time. The augmented reality display system 108 can project the holographic visualization 128 that combines registered images from the first imaging system 102 and the second imaging system 104, allowing the practitioner to simultaneously view multiple perspectives while maintaining hand-eye coordination. The system 100 can be valuable for avoiding injury by identifying structures like blood vessels near the surgical site. The computer system 106 can track the position of interventional instrument 129 in real-time, updating the holographic visualization to show the relationship between the instruments and vital anatomical structures.

In a third exemplary application, the system 100 can be configured to assist with a pain management procedure, where examples include femoral and obturator nerve blocks and ablations, genicular nerve blocks and ablations, medial branch nerve blocks and ablations, vertebroplasty/vertebral augmentations, and epidural injections. For the procedure, which requires both anteroposterior and lateral projections, the system 100 can be utilized in an office setting where a C-arm unit must be repeatedly rotated between views. The anatomical registration device 110 can be positioned on the location of interest to establish spatial correlation between imaging views and the anatomy of the patient.

The computer system 106 can register both fluoroscopic views using the anatomical registration device 110 to establish the spatial correlation. The augmented reality display system 108 can project the holographic visualization 128 combining the anteroposterior and lateral views, spatially aligned with the patient, which enables the practitioner to simultaneously view both perspectives while maintaining hand-eye coordination, militating against the need to repeatedly look away at 2D monitors or rotate the C-arm apparatus between views.

In a fourth exemplary application, the system 100 can be configured to assist with a transcatheter valve procedure and a left atrial appendage (LAA) closure by integrating volume rendering and 4D ultrasound ability. For the procedure, the first imaging system 102 and the second imaging system 104 can be positioned non-coplanar to each other to acquire multiple views of the cardiac structure. The anatomical registration device 110 can establish the spatial correlation between the imaging views and patient anatomy, though for cardiovascular applications, electromagnetic, impedance, and fiberoptic tracking systems can be more appropriate given the internal and flexible nature of the device.

During the procedure, the system 100 can integrate transesophageal (TEE) ultrasound imaging with fluoroscopic views. The augmented reality display system 108 can project the holographic visualization 128 that combines the registered biplanar fluoroscopic views with the TEE images, allowing the practitioner to simultaneously view multiple imaging modalities while maintaining hand-eye coordination. The computer system 106 can track the position of the catheter, stent, and other interventional instrument 129 in real-time, updating the holographic visualization 128 to show the relationship between the interventional instrument 129 and the cardiac structure.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for performing a medical procedure at a location of interest on a patient, comprising:
providing a system for performing the medical procedure on the location of interest of the patient, the system including:
a first imaging system configured to acquire a first image of the location of interest,
a second imaging system disposed non-coplanar to the first imaging system and configured to acquire a second image of the location of interest,
a computer system configured to register the first image and the second image, establish a spatial correlation between the first image, the second image and patient, and generate a holographic visualization combining the first image and the second image, and
an augmented reality display system configured to project the holographic visualization in proximity to the patient, spatially align the first image and the second image with the patient, and enable simultaneous visualization of the first image, the second image, and the patient;
acquiring, by the first imaging system, the first image depicting a first view of the location of interest;
acquiring, by the second imaging system disposed non-coplanar to the first imaging system, the second image depicting a second view of the location of interest, the second view being different than the first view;
registering the first image and the second image to establish a spatial correlation between the first image, the second image, and the patient;
generating the holographic visualization that combines the first image, the second image, and a virtual trajectory for an interventional instrument;
projecting the holographic visualization in proximity to the patient such that the first image and the second image are spatially aligned with the patient,
adjusting orientation and depth of the interventional instrument using the holographic visualization; and
performing the medical procedure guided by the spatially aligned holographic visualization.

2. The method of claim 1, further including a step of tracking a position of the interventional instrument during the medical procedure.

3. The method of claim 1, further including a step of aligning the first imaging system to collect an anteroposterior fluoroscopic image of the location of interest.

4. The method of claim 1, further including a step of aligning the second imaging system to collect a lateral fluoroscopic image of the location of interest.

5. The method of claim 1, wherein the first imaging system includes a first fluoroscopy system.

6. The method of claim 1, wherein the second imaging system includes a second fluoroscopy system.

7. The method of claim 1, wherein the medical procedure includes at least one of an orthopedic procedure, a neurological procedure, a pain management procedure, a cardiovascular procedure, and an otolaryngology procedure.

8. The method of claim 1, further including a step of simulating a surgical plan using a preoperative dataset.

9. The method of claim 8, further including projecting the surgical plan onto the patient using the augmented reality display system.

10. A system for performing a medical procedure on a location of interest of a patient, comprising:
a first imaging system configured to acquire a first image of the location of interest;
a second imaging system disposed non-coplanar to the first imaging system and configured to acquire a second image of the location of interest;
a computer system configured to register the first image and the second image, establish a spatial correlation between the first image, the second image and patient, and generate a holographic visualization combining the first image and the second image; and
an augmented reality display system configured to project the holographic visualization in proximity to the patient, spatially align the first image and the second image with the patient, and enable simultaneous visualization of the first image, the second image, and the patient.

11. The system of claim 10, wherein the first imaging system includes a first fluoroscopy system.

12. The system of claim 11, wherein the second imaging system includes a second fluoroscopy system.

13. The system of claim 12, wherein the second fluoroscopy system is disposed perpendicular to the first fluoroscopy system.

14. The system of claim 10, wherein the first image includes an anteroposterior fluoroscopic image of the location of interest.

15. The system of claim 10, wherein the second image includes a lateral fluoroscopic image of the location of interest.

16. The system of claim 10, wherein the computer system is further configured to track a position of an interventional instrument relative to the first image and the second image and update the holographic visualization to show a tracked position of the interventional instrument in real-time.

17. The system of claim 10, wherein the augmented reality display system includes a head-mounted display configured to project the holographic visualization.

18. The system of claim 10, further including a plurality of imaging systems, each imaging system for acquiring an image.

19. The system of claim 10, wherein the spatial alignment includes aligning an interventional instrument with the first image, the second image, and the patient.

20. A system for performing a medical procedure on a location of interest of a patient, comprising:
a first fluoroscopic system configured to acquire a first fluoroscopic image of the location of interest, the first fluoroscopic image including an anteroposterior fluoroscopic image of the location of interest;
a second fluoroscopic imaging system disposed perpendicular to the first fluoroscopic system and configured to acquire a second fluoroscopic image of the location of interest, the second fluoroscopic image includes a lateral fluoroscopic image of the location of interest;
a computer system configured to register the first fluoroscopic image and the second fluoroscopic image, establish a spatial correlation between the first fluoroscopic image, the second fluoroscopic image and patient, and generate a holographic visualization combining the first fluoroscopic image and the second fluoroscopic image; and
an augmented reality display system configured to project the holographic visualization in proximity to the patient, spatially align the first fluoroscopic image and the second fluoroscopic image with the patient, and enable simultaneous visualization of the first fluoroscopic image, the second fluoroscopic image, and the patient.
